(19) United States
(12) Patent Application Publication
(10) Pub. No.: US 2009/0209891 A1
(43) Pub. Date: Aug. 20, 2009

(54) PRESSURE CONTROLLING DEVICES

(75) Inventors: Noam Gavriely, Haifa (IL); Oren Gavriely, Haifa (IL); Benny Rousso, Rishon-LeZion (IL)

Correspondence Address: MARTIN D. MOYNIHAN d/b/a PRTSI, INC., P.O. BOX 16446, ARLINGTON, VA, 22215, US

(73) Assignee: OHK Medical Devices Ltd., Haifa (IL)

(21) Appl. No.: 12/369,769

(22) Filed: Feb. 12, 2009

Related U.S. Application Data
(60) Provisional application No. 61/064,082, filed on Feb. 14, 2008.

(51) Int. Cl. A61B 17/12

(52) U.S. Cl. 601/134

(57) ABSTRACT

Disclosed is a method for exsanguinating a portion of an extremity. The method comprises, encircling a cross sectional portion of an extremity with at least one exsanguinating pressure inducer, moving one or both of a pressure produced at a pressure application point by the at least one exsanguinating pressure inducer and a portion of the extremity with respect to each other, inducing an exsanguinating pressure on the portion of the extremity to which pressure is applied during the moving, and controlling the exsanguinating pressure to remain within an exsanguinating range of pressure.

### RELATED APPLICATION

This application claims the benefit of priority of U.S. Provisional Patent Application No. 61/064,082 filed on Feb. 14, 2008, the contents of which are incorporated herein by reference.

### FIELD AND BACKGROUND OF THE INVENTION

The present invention, in some embodiments thereof, relates to devices for exsanguination of an extremity and, more particularly, but not exclusively, to devices that apply a constant pressure during exsanguination.

A bloodless extremity during a surgical procedure is highly desirable in helping the surgeon identify tissue layers, anatomic landmarks and biological structures.

To attempt to exsanguinate the extremity and render a bloodless surgical site, a first sterile assistant typically elevates the extremity above the heart while a second sterile assistant wraps an elastic bandage in a spiral fashion from the distal portion of the extremity to proximal to the surgical site.

The elastic wrap drives some of the blood in the extremity proximal to the surgical site and, to prevent a flow of blood during surgery, a pneumatic tourniquet proximal to the surgical site is inflated to a pressure of between 150 and 500 millimeters of mercury (mm Hg). The elastic bandage is then removed and the surgeon makes a first incision into the surgical site.

The first incision, however, is often accompanied by a release of residual blood remaining in the extremity distal to the tourniquet. The residual blood often covers and obstructs visualization of the surgical site. Lavage and copious sponging by a sterile assistant renders a relatively bloodless, but wet, surgical site.

U.S. patent application Ser. No. 10/498,369, now published as U.S. 200510080450A1 (Gavriely) teaches a tubular sock and elastic annulus that roll along the extremity to exsanguinate the extremity.

### SUMMARY OF THE INVENTION

According to one aspect of some embodiments of the invention, there is provided a method for exsanguinating a portion of an extremity, the method comprising, encircling a cross sectional portion of an extremity with at least one exsanguinating pressure inducer, moving one or both of a pressure produced at a pressure application point by the at least one exsanguinating pressure inducer and a portion of the extremity with respect to each other, inducing an exsanguinating pressure on the portion of the extremity to which pressure is applied during the moving, and controlling the exsanguinating pressure to remain within an exsanguinating range of pressure.

According to some embodiments of the invention, the exsanguinating pressure is between 150 and 500 mm Hg.

According to some embodiments of the invention, the exsanguinating range of pressure is plus or minus 25 mm Hg of a chosen exsanguinating pressure between 150 and 500 mm Hg.

According to some embodiments of the invention, the moving comprises moving the at least one exsanguinating pressure inducer with respect to the portion of the extremity.

According to some embodiments of the invention, the method includes twistingly rotating the at least one exsanguinating pressure inducer with respect to the portion of the extremity.

According to some embodiments of the invention, the method includes linearly sliding the at least one exsanguinating pressure inducer with respect to the portion of the extremity.

According to some embodiments of the invention, the linear sliding is applied while the moving is in at least one direction of: proximally and distally with respect to the extremity.

According to some embodiments of the invention, the method includes rotating rotational segments of the at least one exsanguinating pressure inducer with respect to the portion of the extremity.

According to some embodiments of the invention, the method includes orbiting a portion of the at least one exsanguinating pressure inducer around the portion of the extremity.

According to some embodiments of the invention, the method includes serially inducing pressure in at least two sections of the at least one exsanguinating pressure inducer.

According to some embodiments of the invention, the moving comprises moving one portion of the extremity forward into the at least one exsanguinating pressure inducer.

According to some embodiments of the invention, the method includes providing a surface within the at least one exsanguinating pressure inducer.

According to some embodiments of the invention, the method includes rotating a portion of the surface along the one portion of the extremity during the moving.

According to some embodiments of the invention, the method includes increasing the pressure at a constant rate along the surface during the moving.

According to some embodiments of the invention, the method includes surrounding the surface around the one extremity portion and providing a pressure gradient along the surface.

According to some embodiments of the invention, the method includes surrounding the surface around the one extremity portion, reducing pressure along one forward portion of the extremity portion, and maintaining the at least one exsanguinating pressure along one rearward portion of the extremity portion.

According to another aspect of some embodiments of the invention, there is provided a device for exsanguinating a portion of an extremity, the device including a passage having a surface configured to flexibly surround an extremity portion, and one chamber containing extremity-exsanguinating pressure inducing particulate matter surrounding one portion of the passage.

According to some embodiments of the invention, the passage is substantially vertical.

According to some embodiments of the invention, the one chamber comprises at least one vertical wall configured to contain the particulate matter in a pressure gradient along the passage.

According to some embodiments of the invention, the particulate matter comprises fluids from the group of fluids including water, saline, a gel, and mercury.

According to some embodiments of the invention, the particulate matter comprises finite elements suspended in a viscous matrix.

According to some embodiments of the invention, the particulate matter comprises at least two rotatable round elements.

According to some embodiments of the invention, the surface comprises an inner surface of a sleeve.

According to some embodiments of the invention, the particulate matter comprises at least two rotatable round elements.

According to some embodiments of the invention, the at least two rotatable round elements are included in the surface.

According to some embodiments of the invention, the at least two rotatable round elements are axially interconnected.

According to some embodiments of the invention, the sleeve extends beyond the chamber.

According to some embodiments of the invention, one portion of the sleeve extending beyond the chamber comprises two removably connectable edges.

According to a further aspect of some embodiments of the invention, there is provided a device for exsanguinating a portion of an extremity, the device including: a passage having a surface configured to flexibly surround an extremity portion, at least two inflatable bladders at least partially surrounding the surface, including: at least one first inflatable bladder, and at least one second inflatable bladder, and at least one exsanguination pressure-activated valve extending from the at least one first inflatable bladder to the at least one second inflatable bladder.

According to some embodiments of the invention, the pressure-activated valve is configured to open when the pressure in the at least one first inflatable bladder is between 150 and 500 mm Hg.

According to still another aspect of some embodiments of the invention, there is provided a device for exsanguinating a portion of an extremity, the device including: an elongate tube configured to roll into a torus, encircle an annulus of an extremity portion and unroll to apply substantially constant exsanguinating pressures to at least two extremity cross sectional portions, including: one first larger extremity cross section, and one second smaller extremity cross section.

According to some embodiments of the invention, the elongate tube comprises at least two portions: at least one first portion having a smaller average diameter, and at least one section portion having a larger average diameter.

According to some embodiments of the invention, the elongate tube comprises at least two portions: at least one first portion having a lower coefficient of elasticity, and at least one second portion having a higher coefficient of elasticity.

According to still a further aspect of the invention, there is provided a device for exsanguinating a portion of an extremity, the device comprising a looped strap having an exsanguinating loop portion configured to surround an extremity portion and apply an exsanguinating pressure to an extremity, a rotational inducer portion, a rotational inducer coupled to the rotation inducer portion of the strap, the rotational rotation inducer configured to induce rotation in the rotational inducer portion of the strap and a linear movement handle operatively associated with the rotational inducer and configured to facilitate linear movement in the rotational inducer with respect to the extremity.

According to some embodiments of the invention, the linear movement handle is configured to linearly move the rotational inducer in at least one direction with respect to the extremity: proximally and distally.

According to some embodiments of the invention, the exsanguinating pressure is between 150 and 500 mm Hg.

According to some embodiments of the invention, the exsanguinating pressure comprises a range of plus or minus 25 mm Hg of a chosen exsanguinating pressure between 150 and 500 mm Hg.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of orthopedic biomechanics.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1-3 (prior art) show a torus exsanguinating an extremity portion;

FIGS. 4A-4D show a conical sock exsanguination device being applied to a lower extremity, according to embodiments of the present invention;

FIG. 5 shows a tubular sock exsanguination device and lower extremity, according to embodiments of the present invention;

FIGS. 6A-6C show an inflatable-chambered exsanguination device being applied to a lower extremity, according to embodiments of the present invention;

FIGS. 7A-7D show a beaded sock being applied to an upper extremity, according to embodiments of the present invention;

FIGS. 8A-8C and 9A-9C show upper extremity exsanguination in a compression chamber, according to embodiments of the present invention; and

FIGS. 10A-10B, 11A-11B and 12 show continuous strap exsanguination devices being applied to a lower extremity, according to embodiments of the present invention.

### DESCRIPTION OF EMBODIMENTS OF THE INVENTION

The present invention, in some embodiments thereof, relates to devices for exsanguination of an extremity and, more particularly, but not exclusively, to devices that apply a controlled pressure during exsanguination.

In some embodiments, the extremity is exsanguinated within a chosen exsanguinating pressure range that is between 150 and 500 mm Hg. In further embodiments, the exsanguinating pressure range comprises a range of plus or minus 25 mm Hg of a chosen exsanguinating pressure, between the above-noted 150 and 500 mm Hg.

According to the teachings of the present invention there is provided an exsanguination device comprising a tubular sock including sections having varied diameters that correspond to the varied cross-sectional diameters of an extremity.

Tubular sock is rolled into a torus, similar to existing U.S. 200510080450A1 as will be explained below, encircled around a portion of an extremity and unrolled onto the extremity while providing controlled pressure to each portion of the extremity that remains substantially constant throughout extremity.

In alternative embodiments, the sock includes areas that provide differing elasticity pressures, herein coefficients of elasticity, such that as the torus unrolls, the differing elasticity may ensure that the pressure over smaller diameter extremity portions is substantially equal to the pressure over larger diameter extremity portions.

According to further teachings of the present invention there is provided an exsanguination device comprising a tube having multiple inflation chambers configured to surround an extremity portion. The inflation chambers are then inflated serially around the extremity portion. The multiple inflation chambers exsanguinate blood from a first distal extremity section associated with a first inflation chamber, followed by exsanguination of blood from a second, more proximal, extremity portion associated with a second inflation chamber.

The exsanguination pressures applied to the extremity are substantially constant due to the conformation of the various inflation chambers to the cross-sectional diameter of each extremity portion.

According to still further teachings of the present invention there is further provided a pressurized fluid chamber surrounding a portion of a flexible sleeve. As an extremity is passed distally into the flexible sleeve, the surrounding pressure chamber compresses the flexible sleeve, thereby exsanguinating the extremity proximally as the extremity passes in a distal direction into the sleeve.

Optionally, rolling beads are integrated into the flexible chamber in order to facilitate distal movement of the extremity through the pressurized sock.

The exsanguination pressures applied to the extremity are substantially constant due to the conformation of the beaded sleeve and pressure fluid surrounding the sleeve to each extremity portion.

According to still further teachings of the present invention there is provided a continuous strap exsanguination device in which a portion of a continuous strap is looped in a spiral around an extremity. As the device is moved proximally in a linear fashion along the extremity, the spiral rotates around the extremity, thereby exsanguinating the extremity.

By maintaining a specific tension along the continuous strap, for example in embodiments including a tension sensor connected to the continuous strap, constant exsanguination pressure is maintained in the extremity.

The principles and operation of devices configured to control exsanguination pressure along an extremity according to the present invention may be better understood with reference to the drawings and accompanying descriptions.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Referring now to the drawings:

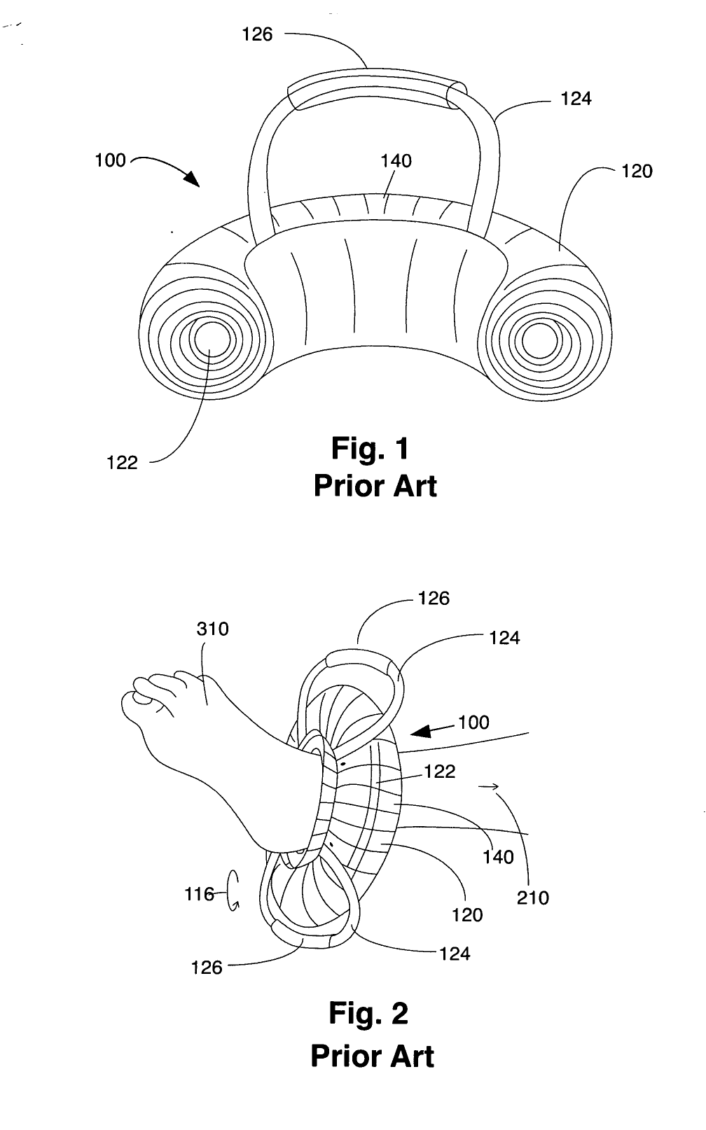

Shown in FIG. 1 is an existing extremity exsanguination assembly 100, shown in cross section, similar to a torus device shown in the above-noted US Application 20050080450A1.

Torus assembly 100 includes an elastic tube 120 folded around a flexible elastic ring 122, herein torus 122, along with application straps 124. According to some embodiments of the invention, torus 122 comprises a single homogenous solid ring of an elastic material, for example rubber.

As seen in FIG. 2, application straps 124 and an associated handle 126 are used to roll torus assembly 100 in a rotational direction 116 along an extremity 310 while elastic tube 120 unrolls from around torus 122 to encircle extremity 310. The rolling motion of torus 122 in rotational direction 116, or the opposite thereof, is referred to herein as twisting rotation.

Torus assembly 100 rolls proximally, torus 122 and a tube roll 140 comprising elastic tube 120 exert pressure on extremity 310 that is higher than the arterial, capillary and venous blood pressure.

Torus has a constant coefficient of elasticity so that initially, when tube roll 140 has a large cross sectional diameter, the pressure exerted on extremity by torus assembly 100 is higher. As tube roll 140 unwinds, the remaining portion of elastic tube 120 around torus 122 is reduced; the inner diameter of tube roll 140 increases and the pressure exerted on extremity 310 is reduced.

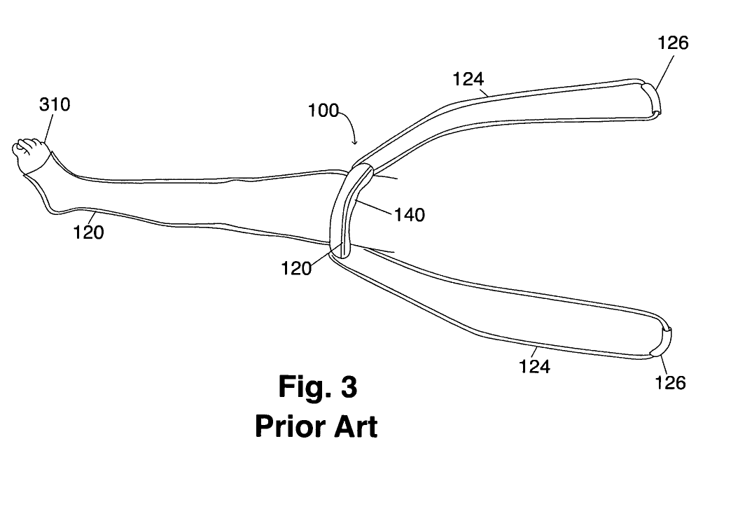

Additionally, as torus assembly 100 moves along thicker areas of extremity 310, for example the thigh, seen in FIG. 3, the pressure exerted by torus assembly 100 may increase drastically.

However, the constrictive pressure generated by torus 122 and elastic tube 120 is higher than the arterial blood pressure yet lower than the pressure that may cause crush injury to the tissues beneath it.

The pressure from torus 122 and the wound remainder of elastic tube 120 squeezes extremity 310 in progression from a distal position to a more proximal position, causing blood to move proximal to torus 122, leaving the distal portion of extremity 310 substantially bloodless.

After torus assembly 100 is located proximal to the surgical site, torus 122 is left in place to prevent blood from entering extremity 310 as would the above-noted tourniquet.

In some embodiments of the present invention, variable diameters along elastic tube 120 may provide effective constant exsanguination pressure. The following embodiment describes just one such variable diameter configuration.

### Conical Sock

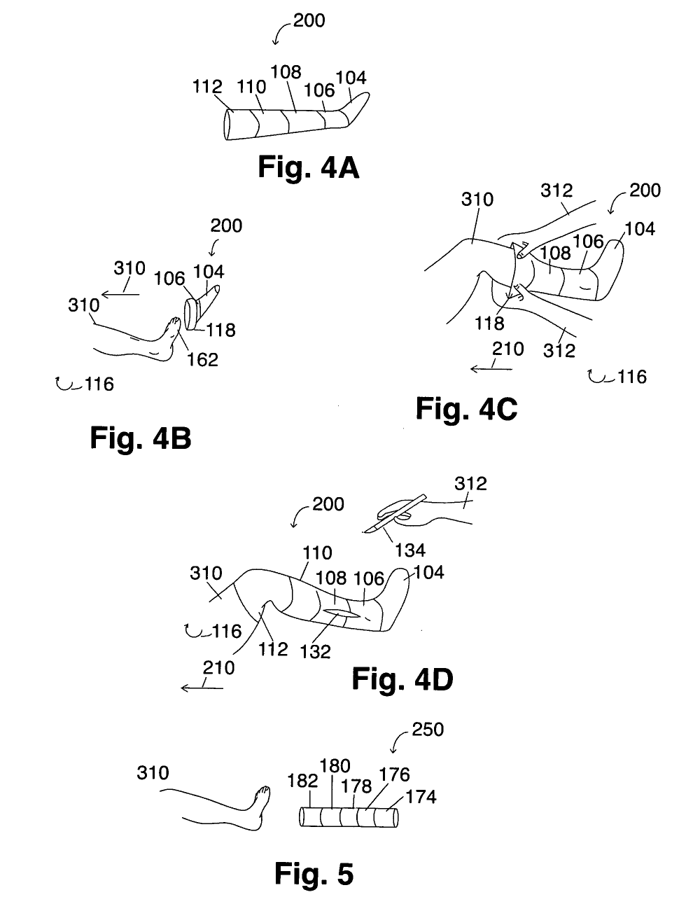

FIG. 4A shows the use of a conical exsanguination device 200 sleeve having a variety of sections of different diameters corresponding to different diameters of the extremity.

Upon application to the extremity, each diameter section provides a constant exsanguination pressure to a given portion of the extremity.

As seen in FIG. 4B a substantial portion of conical exsanguination device 200 has been rolled into a torus 118 which will be placed over extremity 310. The inner diameter of torus 118 has an inner diameter that, including the bulk of sections 104, and 106, for example, creates substantial pressure on the foot, thereby driving blood in a proximal direction 210 as conical exsanguination device 200 is unrolled in direction 116 along a foot 162.

As seen in FIG. 4C, as torus 118 is rolled proximally 210, the inner diameter of torus 118 increases due to less bulk as sections 104, 106, 108, 110, and 112 unroll and make contact with lower extremity 310.

The increasing diameters of sections 104, 106, 108, 110, and 112 maintain a substantially equivalent exsanguination pressure along extremity 310 which, similarly, increases in diameter and bulk in proximal direction 210.

As used herein, any object that applies pressure to an extremity, for example sections 104, 106, 108, 110, and 112, may be alternatively referred to as “pressure inducers”. As used herein, exsanguinate and similar words mean to deprive of or drain of blood.

As seen in FIG. 4D, with section 112 rolled above the knee, lower extremity is substantially exsanguinated. Sections 106 and 108 have been incised with a scalpel 134 to form an incision 132 in a substantially bloodless field.

According to some embodiments of the invention, the diameters of each of sections 104, 106, 108, 110, and 112, and the exsanguination pressure applied thereby, are configured according to the cross sectional diameter of each portion of extremity 310 as well as the amount of tubular sock remaining in wound roll 140 so as to apply substantially constant pressure to all portions of extremity 310.

The inventors have found that sock 200 may provide a more controlled pressure over extremity 310 than existing art U.S. 200510080450A1 noted above, due to the varied diameters of sections 104, 106, 108, 110, and 112.

In other embodiments of the present invention, varied pressure-providing sections of a tube provide effective constant exsanguination pressure. The following embodiment provides just one such varied pressure-providing sections option.

### Tubular Sock

FIG. 5 shows the use of a tubular exsanguination device 250 sleeve having a variety of sections with different stretch ratios, herein coefficients of elasticity, corresponding to different diameters of the extremity.

Upon application to the extremity, the stretch ratio of each section provides a constant exsanguination pressure to a given portion of the extremity.

In embodiments, tubular exsanguination device 250 comprises sections 174, 176, 178, 180, and 182 which have substantially equivalent diameters but variable coefficients of elasticity. During exsanguination of extremity 310 sections 174, 176, provide substantially equivalent circumferential pressure as section 178 which is configured to exsanguinate the ankle.

According to some embodiments of the invention, the elasticity of each of sections 174, 176, 178, 180, and 182, and the exsanguination pressure applied thereby, are configured according to the cross sectional diameter of each portion of extremity 310 as well as the amount of tubular sock 250 remaining in wound roll 140 so as to apply substantially constant pressure to all portions of extremity 310.

The inventors have found that sock 250 may provide a more controlled pressure over extremity 310 than existing art U.S. 200510080450A1 noted above, due to the varied elasticity of sections 174, 176, 178, 180, and 182.

In still further embodiments of the present invention, varied pressure sections are provided through alternative embodiments to the variable elasticity of sections 174, 176, 178, 180, and 182.

The following embodiment provides just one such alternative varied pressure sections option.

### Chambered Inflation Sock

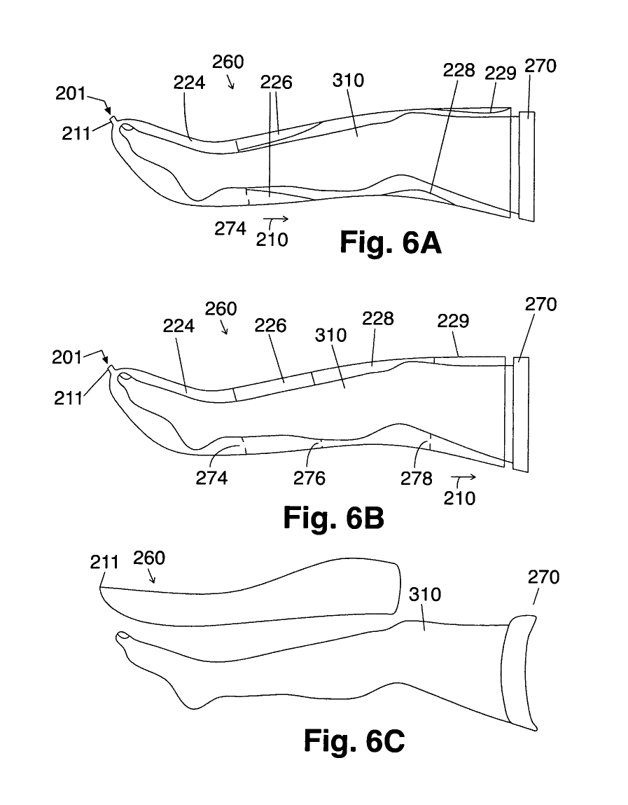

FIGS. 6A-6B show an inflatable-chambered exsanguination sock 260 including serially inflatable compartments, each compartment having an internal inflated diameter corresponding to the diameter of an adjacent portion of the extremity.

Upon inflation, each inflatable section provides a constant exsanguinating pressure to the given extremity portion so that the overall exsanguination pressure throughout the extremity is substantially equivalent.

According to some embodiments of the invention, a fluid 201, for example a gas, is pumped into an inlet 211, filling foot compartment 224 to exsanguinate the foot while blood is pushed out of foot proximally 210. As the pressure in foot compartment 224 increases, a first pressure sensitive valve 274 is triggered by the pressure to open, allowing fluid 201 to enter a calf compartment 226.

As the pressure in calf compartment 226 increases, a second pressure sensitive valve 276 is triggered to open, allowing fluid 201 to enter knee compartment 228.

As the pressure in knee compartment 228 increases, a third pressure sensitive valve 278 is triggered to open, allowing fluid 201 to enter thigh compartment 229. Fully inflated serially inflatable compartments 224, 226, 228, and 229 are configured to compress extremity 310 with between 150 and 500 mm Hg.

In surgical embodiments, a thigh tourniquet 270 is then inflated to between 150 and 500 mm Hg and, as seen in FIG. 6C, inflatable-chambered exsanguination sock 260 is removed from lower extremity 310 and thigh tourniquet 270 maintains a bloodless field.

In embodiments, inflatable-chambered exsanguination device 260 includes serially inflatable compartments 224, 226, 228, and 229 which comprise substantially non-compliant material and maintain a given internal diameter corresponding to a desired exsanguination pressure.

In other embodiments, serially inflatable compartments 224, 226, 228, and 229 comprise substantially compliant materials that may vary in diameter at a given exsanguination pressure. In compliant material serially inflatable compartments 224, 226, 228, and 229 exsanguination device 260 is typically connected to a pressure monitoring device that varies the pressure in compartments 224, 226, 228, and 229 to compensate for compliance, for example stretch in the compliant materials, that may reduce the pressure therein.

Following completion of surgery or when a bloodless field is no longer required, thigh tourniquet 270 is deflated so that blood returns to lower extremity 310.

The inventors have found that inflatable-chambered exsanguination sock 260 may provide a more controlled pressure over extremity 310 than provided in the teachings of U.S. 200510080450A1 noted above, due to final configuration in which serially inflatable compartments 224, 226, 228, and 229 conform to the cross-sectional thickness of various portions of extremity 310 while applying a controlled pressure in each of inflatable compartments 224, 226, 228, and 229.

In still further embodiments of the present invention, varied pressure inflatable sections are replaced with alternative embodiments that provide constant pressure to the extremity. The following embodiment provides just one such alternative constant pressure option.

### Beaded Sleeve

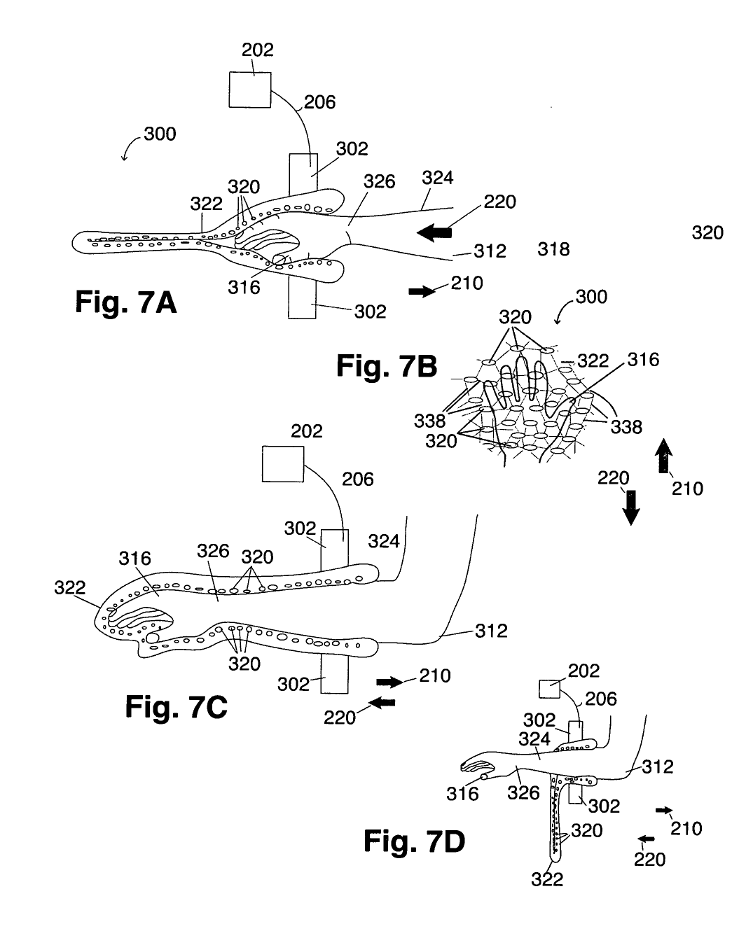

FIG. 7A illustrates the use of a compression chamber exsanguination device 300, optionally an inflation chamber 302 to apply exsanguinations to extremity 312. In embodiments, inflation chamber 302 optionally includes a guide comprising, for example, a beaded sleeve 322 affixed to inflation chamber 302.

Optionally, as extremity 312 is inserted, the pressure is applied to progressive parts of extremity 312. As shown, as a hand 316 presses forward in a distal direction 220, fluid is compressed and/or decompressed within inflation chamber 302. Optionally the fluid maintains a constant pressure in inflation chamber 302 to deliver a continuous circumferential pressure around hand 316 at a range, noted above, between 150 and 500 mm Hg.

During movement of upper extremity, blood is exsanguinated from hand 316 in proximal direction 210 proximal to hand 316.

FIG. 7B shows details of sleeve 322 optionally including multiple bearings 320, or beads 320, which rotate around axes 338 attached to sleeve 322. The rotation of bearings 320 may facilitate movement of hand 316 in distal direction 220.

FIG. 7C shows blood exsanguinated from hand 316 and an arm 324 while contained within sleeve 322.

According to some embodiments of the invention, as seen in FIG. 7D, with the pressure from inflation chamber 302 maintaining blood proximally, sleeve 322 is optionally peeled off hand 316 and arm 324 so that inflation chamber 302 maintains exsanguination of an upper extremity 312.

In still further embodiments of the present invention, alternative embodiments to beaded sleeve 302 are provided. The following embodiment provides just one such alternative embodiment.

### Horizontal Pressure Chamber

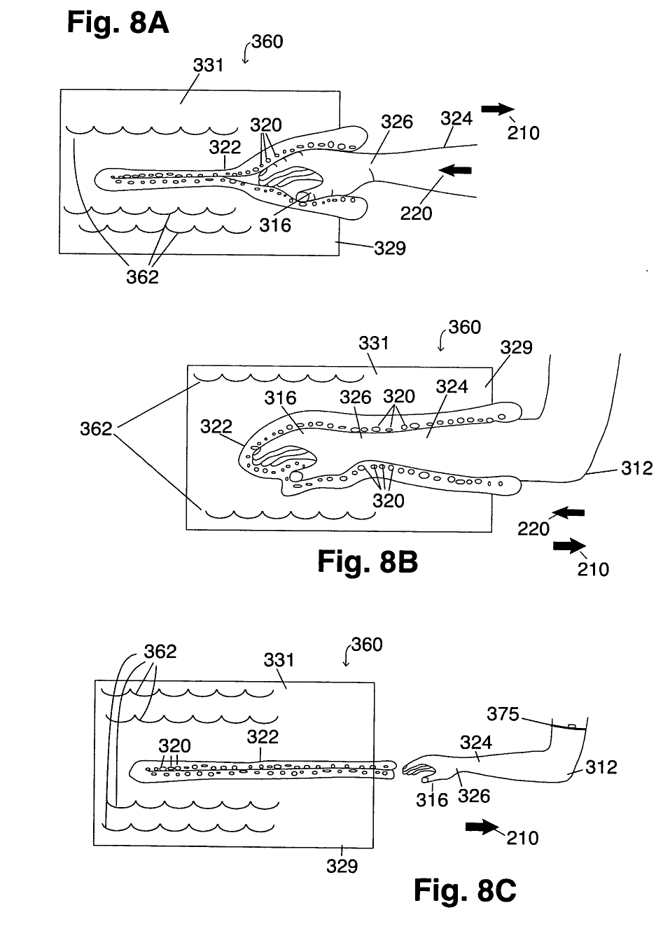

FIG. 8A shows a compression chamber exsanguination device 360 that applies exsanguination pressure to extremity 312. Optionally, compression chamber exsanguination device 360 includes a guide comprising, for example, a beaded sleeve 322 affixed to inflation chamber 302.

As shown, upper extremity 312 enters beaded sleeve 322 within a larger horizontal inflation chamber 331 of a compression chamber exsanguination device 360. As hand 316 presses forward in distal direction 220, a compression material 362, for example a fluid, compresses hand 316, causing the blood to move proximally 210, past hand 316.

FIG. 8B shows blood exsanguinated from hand 316 and arm 324 contained within sleeve 322.

As seen in FIG. 8C, a tourniquet 375 has been applied to maintain exsanguination in hand 316 and arm 324 following removal of upper extremity 312 from horizontal inflation chamber 331.

In still further embodiments of the present invention, alternative embodiments to inflation chamber 300 positioned horizontally are provided. The following embodiment provides just one such alternative horizontal inflation chamber.

### Vertical Pressure Chamber

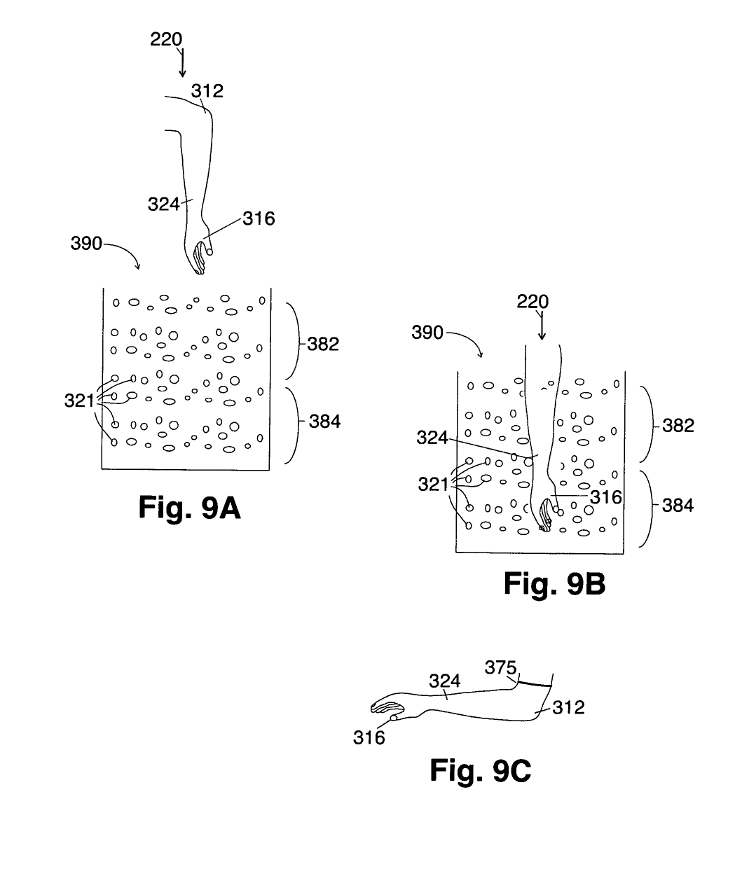

FIG. 9A shows a vertical pressure chamber 390 that applies exsanguination pressure to extremity 312. Optionally, vertical pressure chamber 390 includes a mass of beads that form a passage and roll as hand 316 and arm 324 are pressed distally 220 into vertical pressure chamber 390. The rolling beads form a passage that facilitates the insertion of hand 316 and arm 324.

Alternatively, vertical pressure chamber 390 includes pressure-generating matter 321, for example fluids comprising water, saline, a gel, a viscous suspension of particulate matter, and/or mercury.

In embodiments, generating fluid 321 optionally forms a pressure gradient wherein pressure in a chamber lower section 384 is greater than pressure in a chamber upper section 382.

FIG. 9B shows blood being exsanguinated from hand 316 and arm 324 while in vertical pressure chamber 390. FIG. 9C shows exsanguinated hand 316 and arm 324 with tourniquet 375 applied to maintain exsanguination.

The inventors have found that while vertical pressure chamber 390 provides a pressure gradient that increases between lower section 384 and upper section 382, during insertion of upper extremity, the pressure increase is gradual, thereby possibly providing a more controlled pressure over extremity 312 than existing art U.S. 200510080450A1 noted above which may provide spikes in pressure due to rapidly changing cross-sectional diameters of extremity 312 for example between the wrist and the bicep area of the upper arm.

In still further embodiments of the present invention, constant pressure may be provided by still further alternative embodiments, the following rotating strap exsanginator being just one alternative option.

### Rotating Strap Exsanginator

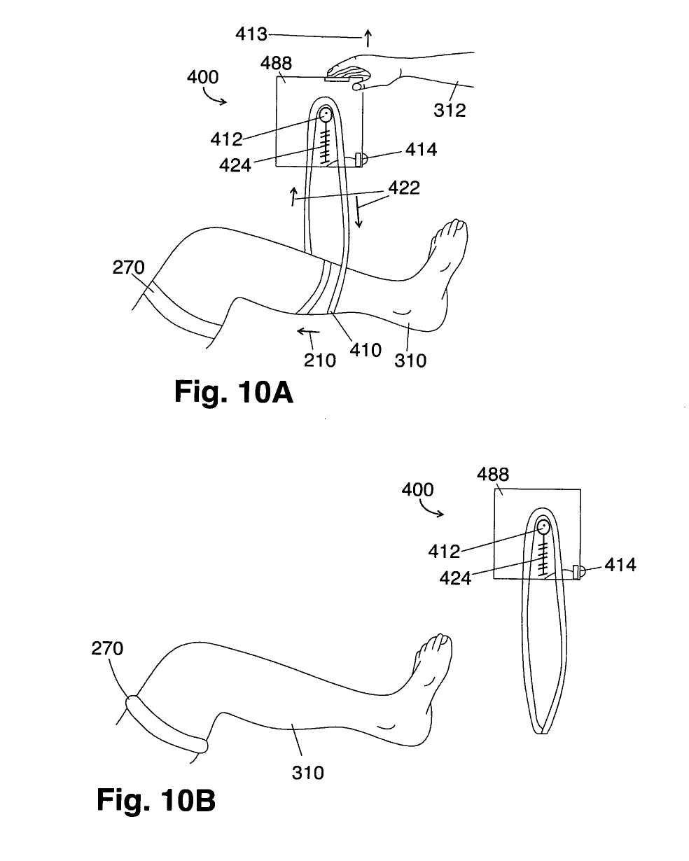

FIG. 10A shows a rotating strap exsanginator 400 that rotates a continuous strap 410 around extremity 310 while moving linearly proximally along extremity 310, thereby exsanguinating extremity 310. Optionally, continuous strap 410 is held in a manner that maintains constant exsanguination pressure around all parts of extremity 310.

As shown, a portion of a continuous strap 410 is looped around lower extremity 310 and rotated around extremity 310. Rotating strap exsanginator 400 is held manually above lower extremity 310 while exsanguinating pressure provided by continuous strap 410 is maintained by a tension transducer 424.

According to some embodiments of the invention, continuous strap 410 is rotated in directions 422 by a motorized wheel 412 within an encasement 488.

While elevating in a direction 413, the operator moves rotating strap exsanginator 400 proximally 210 to exsanguinate the foot and calf of lower extremity 310 as a result of rotating motion 422 of the portion of continuous strap 410 that is in contact with extremity 310.

According to some embodiments of the invention, rotating strap exsanginator 400 may include an emitter 414 that aids in ensuring an operator of appropriate exsanguination pressure.

Should the operator move rotating strap exsanginator 400 too close or too far from extremity 310 to the point that tension transducer 424 cannot compensate and maintain tension at the proper level, an audio and/or visual signal is emitted from emitter 414 signaling the operator to move rotating strap exsanginator 400 closer or further from lower extremity 310.

As seen in FIG. 10B, following exsanguination, tourniquet 270 is applied to an area proximal to the exsanguinated area and rotating strap exsanginator 400 is removed from extremity.

### Mechanized Hoop Exsanginator

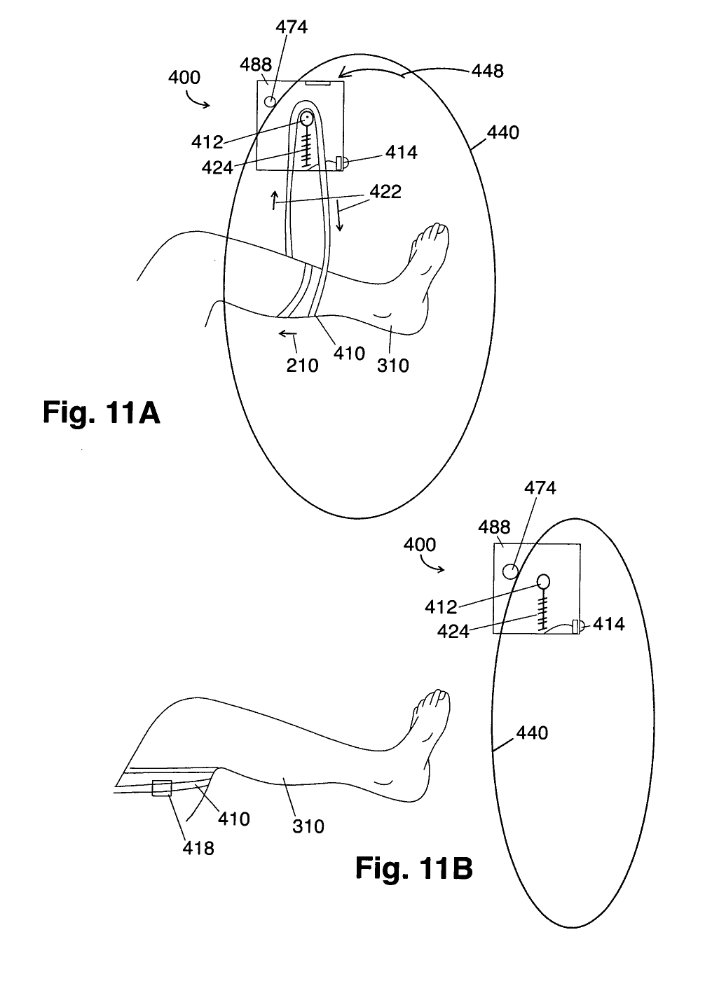

FIGS. 11A-11B show a motorized rotating strap exsanginator 400 that rotates continuous strap 410. Alternatively, strap exsanginator 400 is rotated along a hoop 440 during linear movement along extremity 310.

In embodiments, as seen in FIG. 11A, rotating strap exsanginator 400 includes a motor 474 and moves around extremity 310 in a clockwise direction 448 along hoop 440 under the power of motor 474. Simultaneously, the operator moves hoop 440 proximally 210 to exsanguinated lower extremity 310.

According to some embodiments of the invention, as seen in FIG. 11B, continuous strap 410 is removed from rotating strap exsanginator 400 and secured with a fastener, for example a clamp 418, thereby maintaining the exsanguination of extremity 310. In some embodiments, hoop 440, rather than encasement 488, is rotated around extremity 310.

### Manual Hoop Exsanginator

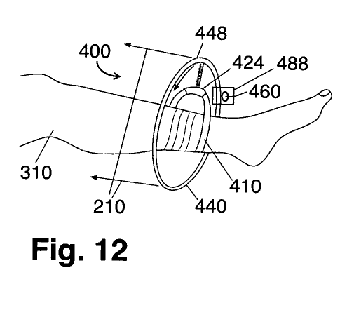

FIG. 12 shows rotating strap exsanginator 400 that is powered to cause rotation of continuous strap 410 and, optionally, rotational motion of a hoop 440 during linear movement along extremity 310.

In embodiments, rotating strap exsanginator 400 includes a mechanized wheel 460 within encasement 488 that rotates hoop 440.

According to some embodiments of the invention, the operator holds encasement 488. Hoop 440 rotates in clockwise direction 448 while the operator moves rotating strap exsanginator 400 proximally 210, thereby exsanguinating extremity 310.

The inventors have found that rotating strap exsanginator embodiments 400 may provide a more controlled pressure over extremity 312 than prior art noted above due to continuous strap 410 conforming to the cross-sectional thickness of various portions of lower extremity 310 in addition to the various embodiments that ensure constant pressure.

It is expected that during the life of this patent many relevant materials and designs for torus exsanguination devices will be developed and the scope of the term “torus exsanguination devices” is intended to include all such new technologies a priori.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method for exsanguinating a portion of an extremity, the method comprising:
i) encircling a cross sectional portion of an extremity with at least one exsanguinating pressure inducer;
ii) moving one or both of a pressure produced at a pressure application point by said at least one exsanguinating pressure inducer and a portion of said extremity with respect to each other;
iii) inducing an exsanguinating pressure on the portion of the extremity to which pressure is applied during said moving; and
iv) controlling said exsanguinating pressure to remain within an exsanguinating range of pressure.

2. The method according to claim 1, wherein said exsanguinating pressure is between 150 and 500 mm Hg.

3. The method according to claim 2, wherein said exsanguinating range of pressure is plus or minus 25 mm Hg of a chosen exsanguinating pressure between 150 and 500 mm Hg.

4. The method according to claim 1, wherein said moving comprises moving said at least one exsanguinating pressure inducer with respect to said portion of said extremity.

5. The method according to claim 4, including twistingly rotating said at least one exsanguinating pressure inducer with respect to said portion of said extremity.

6. The method according to claim 4, including linearly sliding said at least one exsanguinating pressure inducer with respect to said portion of said extremity.

7. The method according to claim 6, wherein said linearly sliding is applied while said moving is in at least one direction of:
proximally; and
distally,
with respect to said extremity.

8. The method according to claim 4, including rotating rotational segments of said at least one exsanguinating pressure inducer with respect to said portion of said extremity.

9. The method according to claim 4, including orbiting a portion of said at least one exsanguinating pressure inducer around said portion of said extremity.

10. The method according to claim 1, including serially inducing pressure in at least two sections of said at least one exsanguinating pressure inducer.

11. The method according to claim 1, wherein said moving comprises moving one portion of said extremity forward into said at least one exsanguinating pressure inducer.

12. The method according to claim 11, including providing a surface within said at least one exsanguinating pressure inducer.

13. The method according to claim 12, including rotating a portion of said surface along said one portion of said extremity during said moving.

14. The method according to claim 12, including increasing said pressure at a constant rate along said surface during said moving.

15. The method according to claim 12, including surrounding said surface around said one extremity portion and providing a pressure gradient along said surface.

16. The method according to claim 12, including:
a) surrounding said surface around said one extremity portion;
b) reducing pressure along one forward portion of said extremity portion; and
c) maintaining said at least one exsanguinating pressure along one rearward portion of said extremity portion.

17. A device for exsanguinating a portion of an extremity, the device comprising:
i) a passage having a surface configured to flexibly surround an extremity portion; and
ii) one chamber containing extremity-exsanguinating pressure inducing particulate matter surrounding one portion of said passage.

18. The device according to claim 17, wherein said passage is substantially vertical.

19. The device according to claim 18, wherein said one chamber comprises at least one vertical wall configured to contain said particulate matter in a pressure gradient along said passage.

20. The device according to claim 17, wherein said particulate matter comprises fluids from the group of fluids comprising water, saline, a gel, and mercury.

21. The device according to claim 17, wherein said particulate matter comprises finite elements suspended in a viscous matrix.

22. The device according to claim 17, wherein said particulate matter comprises at least two rotatable round elements.

23. The device according to claim 17, wherein said surface comprises an inner surface of a sleeve.

24. The device according to claim 23, wherein said inner surface of said sleeve includes at least two rotatable round elements.

25. The device according to claim 24, wherein said at least two rotatable round elements are axially interconnected.

26. The device according to claim 24, wherein said sleeve extends beyond said chamber.

27. The device according to claim 26, wherein one portion of said sleeve extending beyond said chamber comprises two removably connectable edges.

28. A device for exsanguinating a portion of an extremity, the device comprising:
i) a passage having a surface configured to flexibly surround an extremity portion;
ii) at least two inflatable bladders at least partially surrounding said surface, comprising:
at least one first inflatable bladder; and
at least one second inflatable bladder; and
iii) at least one exsanguination pressure-activated valve extending from said at least one first inflatable bladder to said at least one second inflatable bladder.

29. The device according to claim 28, wherein said pressure-activated valve is configured to open when the pressure in said at least one first inflatable bladder is between 150 and 500 mm Hg.

30. A device for exsanguinating a portion of an extremity, the device comprising:
i) an elongate tube rolled into a torus, sized to encircle an annulus of an extremity portion and apply a pressure that is sufficient to exsanguinate said extremity portion;
ii) a first tube section with one first larger extremity cross section; and
iii) a second tube section with one second smaller extremity cross section.

31. The device according to claim 30, wherein said elongate tube comprises at least two portions:
a) at least one first portion having a smaller average diameter; and
b) at least one second portion having a larger average diameter.

32. The device according to claim 30, wherein said elongate tube comprises at least two portions:
a) at least one first portion having a lower coefficient of elasticity; and
b) at least one second portion having a higher coefficient of elasticity.

33. A device for exsanguinating a portion of an extremity, the device comprising:
i) a looped strap having:
an exsanguinating loop portion configured to surround an extremity portion and apply an exsanguinating pressure to an extremity;
a rotational inducer portion;
ii) a rotational inducer coupled to said rotation inducer portion of said strap, said rotation inducer configured to induce rotation in said rotational inducer portion of said strap; and
iii) a linear movement handle operatively associated with said rotational inducer and configured to facilitate linear movement in said rotational inducer with respect to said extremity.

34. The device according to claim 33, wherein said linear movement handle is configured to linearly move said rotational inducer in at least one direction, with respect to said extremity of:
proximally; and
distally.

35. The device according to claim 33, wherein said exsanguinating pressure is between 150 and 500 mm Hg.

36. The device according to claim 35, wherein said exsanguinating pressure comprises a range of plus or minus 25 mm Hg of a chosen exsanguinating pressure between 150 and 500 mm Hg.
